(19) United States
(12) Patent Application Publication
(10) Pub. No.: US 2005/0247155 A1
(43) Pub. Date: Nov. 10, 2005

(54) Bending structure of bendable opener

(76) Inventor: Kuo-Chen Liu, Taichung Hsien (TW)

Correspondence Address: KUO-CHEN LIU, 235 CHUNG-HO BOX 8-24, TAIPEI HSIEN, 235, TW

(21) Appl. No.: 10/837,584

(22) Filed: May 4, 2004

(51) Int. Cl. A61B 1/00

(52) U.S. Cl. 74/490.05

(57) ABSTRACT

A bending structure of a bendable opener comprises a bending portion having a combining tube and a connecting tube. The combining tube and the connecting tube have respective inclined surfaces. The two inclined surfaces are combined and the combining tube is rotatable with respect to the connecting tube. A driving portion is installed in the combining tube and a driven portion is installed in the connecting tube. A driven portion serves to connect the driving portion to the driven portion and is installed in the combining tube and connecting tube. By above structure, to adjust the position of the combining tube corresponding to the connecting tube will cause the opener to be a straight opener or a bending opener.

### FIELD OF THE INVENTION

The present invention relates to bendable openers, and particular to a bendable opener which can be adjusted to be as a straight type opener or a bended opener so as to be used in various places as required.

### BACKGROUND OF THE INVENTION

A bendable opener has a bending body so that the driving tool, for example, an electromotive tool or a pneumatic tool, is bended with respect to the opener head. Thereby, the bendable opener can be used to work in a narrow space, in that the operation of the opener cannot be successful.

However, in many applications, the straight type opener is more successful. As a result, the users frequently carry two kinds of openers, but this is inconvenient for the users.

### SUMMARY OF THE INVENTION

Accordingly, the primary object of the present invention is to provide a bendable opener which can be adjusted to be as a straight type opener.

To achieve object, the present invention provides a bending structure of a bendable opener which comprises a bending portion having a combining tube and a connecting tube. The combining tube and the connecting tube have respective inclined surfaces. The two inclined surfaces are combined and the combining tube is rotatable with respect to the connecting tube. A driving portion is installed in the combining tube and a driven portion is installed in the connecting tube. A driven portion serves to connect the driving portion to the driven portion and is installed in the combining tube and connecting tube. By above structure, to adjust the position of the combining tube corresponding to the connecting tube will cause the opener to be a straight opener or a bending opener.

The various objects and advantages of the present invention will be more readily understood from the following detailed description when read in conjunction with the appended drawing.

### BRIEF DESCRIPTION OF THE DRAWINGS

FIG. 1 is exploded perspective view showing the structure of the bendable opener according to the present invention.

FIG. 2 is a schematic cross view of the combining tube of the bendable opener of the present invention.

FIG. 3 is a schematic cross view of the connecting tube of the bendable opener according to the present invention.

FIG. 4 is a perspective view of the bendable opener of the present invention.

FIG. 5 is a cross section view about the bendable opener of the present invention.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

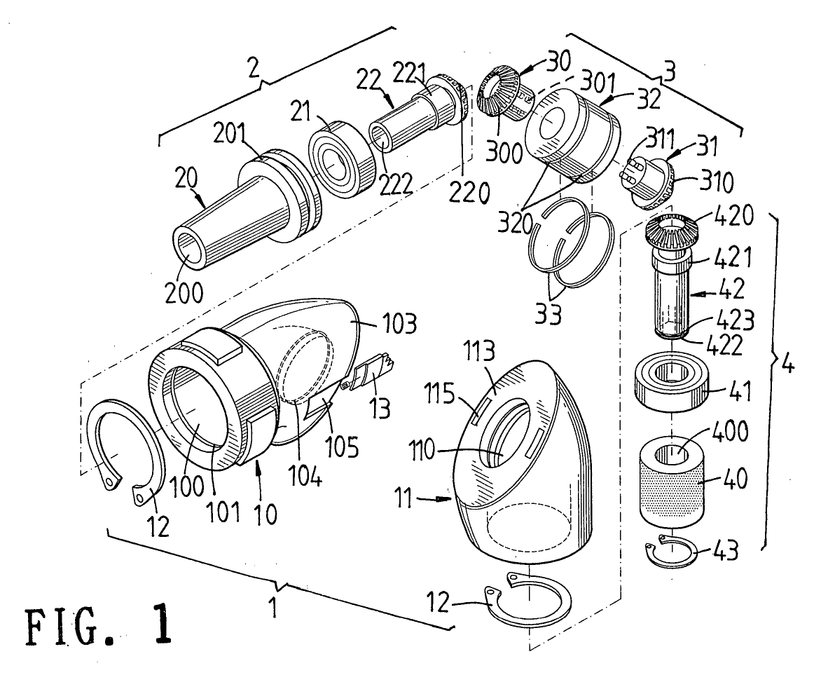

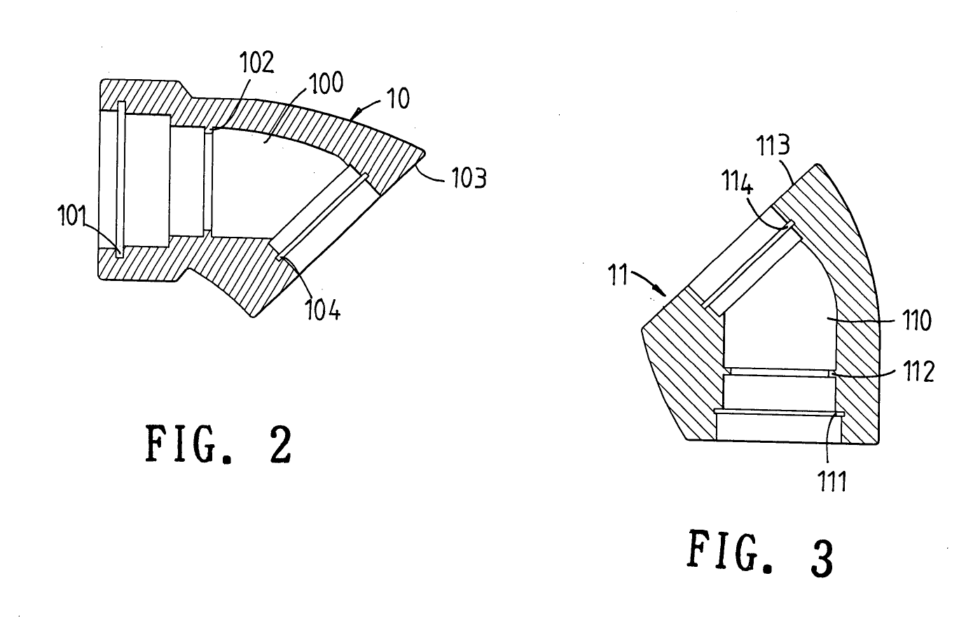

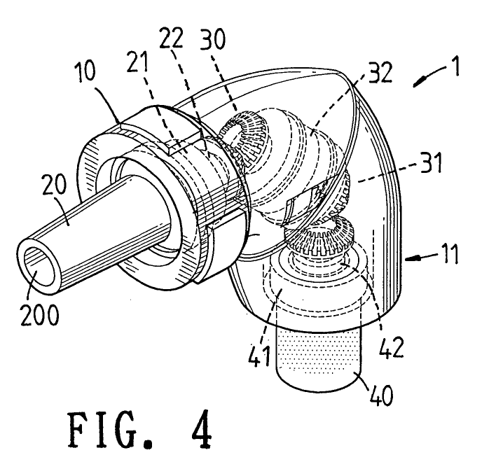

With reference to FIGS. 1 to 4, the bendable opener of the present invention is illustrated. The bendable opener of the present invention mainly comprises a bending portion 1, a driven portion 3 and a driven portion 4.

The bending portion 1 has the following elements.

A combining tube 10 has a receiving space 100 therein. One end of the receiving space 100 is a large annular groove 101 buckling with a large C ring 12. A flange 102 is formed on an inner wall of the combining tube 10 and near the large annular groove 101. Another end of the combining tube 10 is formed with an inclined surface 103 of 45 degrees. A middle annular groove 104 is formed in the inner wall of the combining tube 10 near the inclined surface 103. A receiving chamber 105 is formed on an outer wall of the combining tube 10 near the inclined surface 103. A buckle 13 is installed in the receiving chamber 105.

A connecting tube 11 is installed with a receiving space 110 therein. One inner wall of one end of the receiving space 110 near an opening of the connecting tube 11 has a large annular groove 111 for locking a large C ring 12. A flange 112 is formed at an inner wall of the connecting tube 11 near the large annular groove 111. Another end of the receiving space 110 is formed with an inclined surface 113 with an inclined angle of 45 degrees. An inner wall of the connecting tube 11 near the inclined surface 113 is formed with a middle annular groove 114. Two buckling recesses 115 are formed on the inclined surface 113 and near the edge of the inclined surface 113 for buckling the buckle 13. The driving portion 2 has a driving tube 20, a sleeve 21 and a driving rod 22. An inner space of the driving tube 20 is formed with a cylindrical space 200. A buckling flange 201 is formed at an outer periphery of the driving tube 20. The driving rod 22 can be tightly engaged to the sleeve 21. One end of the driving rod 22 is formed with an opening 222 for receiving an electromotive tools (not shown) or a pneumatic tool (not shown). An outer diameter of the driving rod 22 is slightly smaller than the cylindrical space 200 of the driving tube 20. The remaining portion of the sleeve 21 inserted into the driving rod 22 can be received in the driving tube 20. Another end of the driving rod 22 is installed with an umbrella gear 220. A buckling flange 221 is formed on an outer side of the driving rod 22 near the umbrella gear 220.

The driven portion 3 has one end connected to the driving portion 2 and has another end connected to the driven portion 4. The driven portion 3 has a driving unit 30, a driven unit 31, a gear sleeve 32 and two middle C rings 33. One end of the driving unit 30 has an umbrella gear 300 which is engagable with the umbrella gear 220 of the driving rod 22 and is driven by the driving rod 22. Another end of the driving unit 30 is formed with a plurality of recesses 301. One end of the driven unit 31 has an umbrella gear 310 and another end of the driven unit 31 has a plurality of posts 311 corresponding to the recesses 301 of the driving unit 30 for coupling to the recesses 301. The driven unit 31 is driven by the driving unit 30. The gear sleeve 32 serves to receive the portions of the driving unit 30 and the driven unit 31 other than the umbrella gears 300, 310. An outer side of the gear sleeve 32 is formed with two middle annular grooves 320 for buckling two middle C rings-33.

The driven portion 4 includes a driven tube 40, a bearing cover 41, a driven rod 42, and a small C ring 43. An inner side of the driven rod 42 is formed with a cylindrical space 400. The driven rod 42 is tightly engaged with the bearing cover 41. One end of the driven rod 42 has a receiving opening 422 for receiving a head of an opener (not shown). An outer edge of the receiving opening 422 has a smaller annual groove 423. An outer diameter of the driven rod 42 is slightly smaller than the diameter of the cylindrical space 200. Thereby, the remaining portion of the driven rod 42 after inserted into the bearing cover 41 can be received in the driven rod 42. Another end of the driven rod 42 is installed with an umbrella gear 420 for engaging the umbrella gear 310 of the driven unit 31 so as to be driven by the driven unit 31. A flange 412 is formed at an outer side of the driven rod 42 near the umbrella gear 420.

In assembly of the present invention, the driving rod 22 is received into the sleeve 21 and the driving tube 20 so that the sleeve 21 resists against the flanges 201, 221 of the driving tube 20 and the driving rod 22, respectively to complete the assembly of the driving portion 2. The driving rod 22 is placed into the receiving space 100 of the combining tube 10. The sleeve 21 of the driving portion 2 resists against the flange 102 of the receiving space 100. Then the large C ring 12 serves to buckle the large annular groove 101 so that the driving portion 2 is limited in between the flange 102 of the combining tube 10 and the large C ring 12. The driving unit 30 is inserted into one end of the gear sleeve 32. Then the driving unit 30 and the gear sleeve 32 are placed into the receiving space 100 of the combining tube 10 from the inclined surface 103 of the combining tube 10 so that the driving unit 30 is engaged with the umbrella gears 300, 220 of the driving rod 22. Moreover, the axial line of the umbrella gear 300 of the driving unit 30 is parallel to the normal line of the inclined surface 103 of the combining tube 10. Then the middle C rings 33 are buckled to the middle annular grooves 104, 420 of the combining tube 10 and the gear sleeve 32, respectively. Thereby, the gear sleeve 32 is assembled to the combining tube 10. The posts 311 of the driven unit 31 are inserted into the recesses 301 of the driving unit 30. The driven unit 31 and the gear sleeve 32 are received into the receiving space 110 of the connecting tube 11 so that the axial line of the umbrella gear 410 of the driven unit 31 is in parallel to the normal line of the inclined surface 113 of the connecting tube 11. Then middle C rings 33 are buckled to the middle annular grooves 114, 320 of the gear sleeve 32. Another end of the gear sleeve 32 is assembled to the connecting tube 11. By the buckling effect of the two middle C rings 33, the inclined surface 103 of the combining tube 10 and the inclined surface 113 of the connecting tube 11 will be combined. Thereby, the buckles 13 of the combining tube 10 will be buckled in the buckling recesses 115. The driven rod 42 is placed into the bearing cover 41 and the driven tube 40 so that the small annular groove 423 at a front end of the driven rod 42 protrudes out of the driven tube 40 so that the bearing cover 41 resists between an upper end of the driven tube 40 and the flange 421 of the driven rod 42. Then the small C ring 43 is received in the small annular groove 423 of the driven rod 42 so as to combine the driven rod 42 with the driven tube 40. Therefore, the assembly of the driven portion 4 is complete. Then the driven rod 42 is inserted into the receiving space 110 of the connecting tube 11. Then the bearing cover 41 of the driven portion 4 resists against the flange 102 of the receiving space 110. Then the large C ring 12 is received into the large annular groove 101 of the connecting tube 11 so that the driven portion 4 is limited in the receiving space 110 of the connecting tube 11. Thereby, the assembly of the present invention is complete.

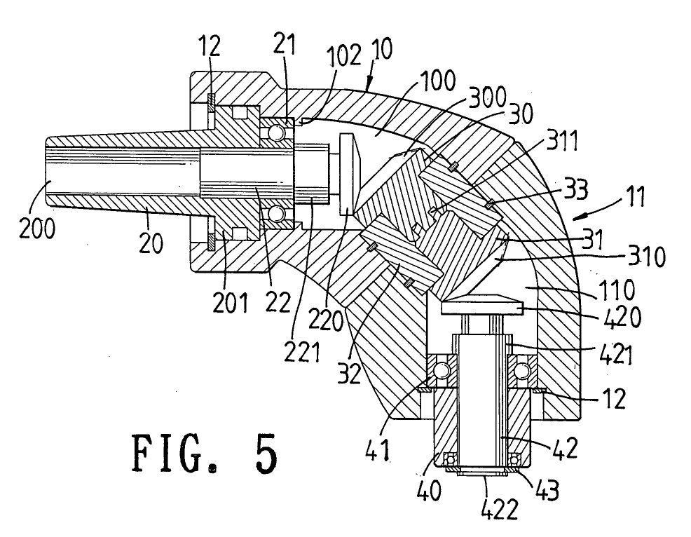

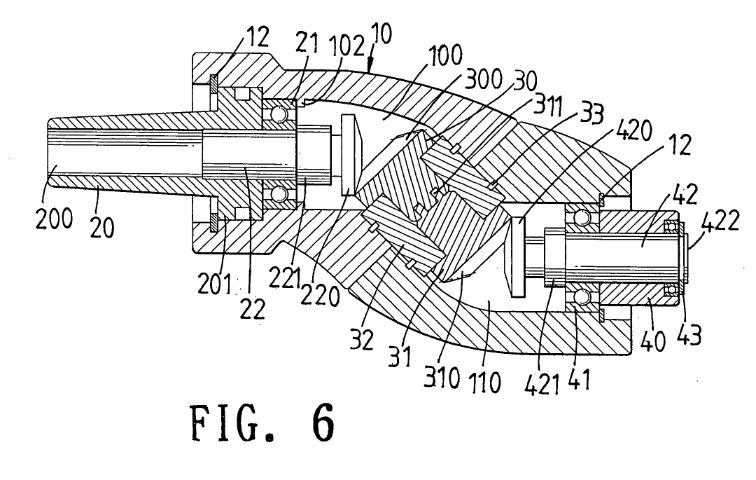

In use of the present invention, referring to FIGS. 4 to 6, the opening 222 of the driving rod 22 of the driving portion 2 is connected to an electromotive or pneumatic tool. The opening 422 of the driven rod 42 of the driven portion 4 can receive an opener head. When the driving rod 22 rotates, the driven portion 3 can drive the driven rod 42 of the driven portion 4 by the umbrella gears 300, 310 to cause the opener head to be received in the opening 422 of the driven rod 42. The driving portion 2 connected to the electromotive or pneumatic tool is vertical to the opener head in the driven portion 4. Thereby, the operation can be performed in some locations having obstacles.

If it is desired to adjust the present invention as a straight means, the buckles 13 of the combining tube 10 of the bending portion 1 is twisted inwards so that the front end thereof is separated from the buckling recess 115 on the inclined surface 113 of the connecting tube 11. Then the connecting tube 11 can be rotated. When the combining tube 10 is rotated through 180 degrees with respect to the connecting tube 11. The buckle 13 of the combining tube 10 are overlapped with another buckling recess 115 of the inclined surface 113 of the connecting tube 11. The buckle 13 will protrude into the buckling recess 115 so as to prevent the combining tube 10 of the bending portion 1 from rotating with respect to the connecting tube 11. FIG. 6 shows the structure of a straight type opener. Since the driven portion 3 is at the rotation axial center, the position of the driven portion 3 is unchanged after the rotation of the bending portion 1 and thus is engaged to the driving rod 22 of the driving portion 2 so as to drive the driven rod 42 of the driven portion 4 and the opener head on the driven rod 42.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A bending structure of a bendable opener comprising:
a bending portion comprising:
a combining tube; one end of the combining tube being chamfered to has an inclined surface with a 45 degree to an axis of the combining tube;
a connecting tube; one end of the connecting tube being chamfered to have an inclined surface with a 45 degree to an axis of the connecting tube; the inclined surface of the combining tube being connected to the inclined surface of the connecting tube; the connecting tube being rotatable with respect to the combining tube;
so that at one connecting position, the combining tube is vertical to the connecting tube; and at another connecting position, the combining tube and the connecting tube are at the same line;
a driving portion receiving in the combining tube; one end of the driving portion near the inclined surface of the combining tube has an umbrella gear; and another end of the driving portion being installed with a driving rod for connecting an electromotive tool and thus being driven by a driving tool; when the driving rod rotates, the umbrella gear of the driving portion will rotate therewith;
a driven portion having umbrella gears at two ends; the two umbrella gears of the driven portion moving interactively; the driven portion being installed between the combining tube and the connecting tube; axial lines of the two umbrella gears being parallel to normal lines of the two inclined surfaces of the combining tube and the connecting tube, respectively; one umbrella gear of the driven portion being engaged to the umbrella gear of the driving portion;
a driven portion being received in the connecting tube and one end of the driven portion having one umbrella gear near the inclined surface of the connecting tube; the umbrella gear of the driven portion being engaged to the other umbrella gear of the driven portion; the driven portion rotating with the driven portion; another end of the driven portion being installed with a driven rod; the driven rod interacting with the umbrella gear of the driven portion; and the driven rod having an opening for receiving an opener head.

2. The bendable opener as claimed in claim 1, wherein the driving tool is an electromotive tool.

3. The bendable opener as claimed in claim 1, wherein the driving tool is a pneumatic tool.

4. The bendable opener as claimed in claim 1, wherein a buckle is installed in the connecting tube; and at least one buckling recess is formed in the combining tube.

5. The bendable opener as claimed in claim 1, wherein a buckle is installed in the combining tube; and at least one buckling recess is formed in the connecting tube.
